(19) United States
(12) Patent Application Publication
(10) Pub. No.: US 2025/0325317 A1
(43) Pub. Date: Oct. 23, 2025

(54) CABLE ASSEMBLY

(71) Applicant: HUBER+SUHNER AG, HERISAU (CH)

(72) Inventor: Maurizio DE CET, Goldach (CH)

(73) Assignee: HUBER+SUHNER AG, HERISAU (CH)

(21) Appl. No.: 18/849,946

(22) PCT Filed: Mar. 22, 2023
(86) PCT No.: PCT/EP2023/057265

§ 371 Date: Sep. 23, 2024

(30) Foreign Application Priority Data
Mar. 24, 2022 (CH) CH000322/2022

(51) Int. Cl. A61B 18/00; H01B 11/18

(52) U.S. Cl. CPC A61B 18/00; H01B 11/1813; H01B 11/1895; A61B 2018/00005; A61B 2018/00095; A61B 2018/00178; A61B 2018/00577; A61B 2018/0091

(57) ABSTRACT

A cable assembly (1) for medical radio frequency or microwave ablation. The cable assembly includes a coaxial cable (2) with an inner conductor (3), encompassed by a dielectric layer (4), encompassed by an outer conductor (5). The cable assembly (1) further includes a first cooling line (6) and a thermal conductive layer (7), which in a cross-sectional view encompasses the coaxial cable (2) and the first cooling line (6) in a circumferential manner. The thermal conductive layer (7) is configured to reduce a thermal peak caused by the coaxial cable (2) when transferring energy, by distributing thermal energy around the coaxial cable (2) and the first cooling line (6) in a circumferential manner.

### BACKGROUND OF THE INVENTION

### Field of the Invention

The present disclosure relates to a cable assembly and an ablation device for use in medical treatments comprising such a cable assembly, in particular for ablation in medical treatments e.g. by Radio Frequency or Microwaves applied in a local treatment.

### Discussion of Related Art

EP 3549544 A1 published by Neuwave Medical Inc. on 9 Oct. 2019, relates to comprehensive systems, devices and methods for delivering energy to tissue for a wide variety of applications, including medical procedures like tissue ablation, resection, cautery, vascular thrombosis, treatment of cardiac arrhythmias and dysrhythmias, electro surgery, tissue harvest. In certain embodiments, systems, devices, and methods are provided for treating a tissue region like a tumor through application of energy. The disclosed coaxial cable impedance is optimized to reduce losses to a value above 50 Ohms.

CA 2635316 A1 published by UK Investments Associates LLC on 12 Jul. 2007, relates to a dipole microwave applicator which emits microwave radiation into tissue to be treated. The applicator is formed from a thin coax cable having an inner conductor surrounded by an insulator, which is surrounded by an outer conductor. A portion of the inner conductor extends beyond the insulator and the outer conductor. A ferrule at the end of the outer conductor has a step and a sleeve that surrounds a portion of the extended inner conductor. A tuning washer is attached to the end of the extended inner conductor. A dielectric tip encloses the tuning washer, the extended inner conductor, and the sleeve of the ferrule. The sleeve of the ferrule and the extended inner conductor operate as the two arms of the dipole microwave antenna. The tuning washer faces the step in the ferrule, and is sized and shaped to cooperate with the step in balancing and tuning the applicator.

EP 3511046 A1, EP 3735928 A1, WO 03024309 A2 relate to cooling structures built with multi-lumen tubing around the cable with gas and/or liquid flowing intermittent forward and backward in neighbored lumens.

### SUMMARY OF THE INVENTION

In particular, for the treatment of cancer with an ablation process, intense signals in the form of radio frequency (RF) or microwaves (MW) are transferred from a generator to a catheter. The catheter is usually inserted into a patient for the treatment of tumor cells by local heating in an ablation process. The energy is thereby emitted from the tip of the catheter to obliterate the tumor cells in the body part. To destroy these tumor cells, small regions of tissue need to be treated. To obliterate the cancerous tissue, the waves e.g. radio frequency (RF) or microwaves (MW) are transmitted from a generator to the catheter tip. The transmitted waves cause a local heating of the tissue above a level which causes the tumor cells to be obliterated. For transferring the high energy necessary to locally heat up the tissue, the treatment is conducted with an ablation device, which comprises a cable assembly, connecting the generator to the actual catheter which is inserted into the patient.

One of the problems to be solved by the present disclosure is to keep the outer temperature of a cable assembly below a critical temperature level that would e.g. cause damage to human tissue or skin which comes in contact with the cable assembly.

An ablation device according to the disclosure typically comprises a handle for the operator with a thereto attached catheter. The handle with the usually therein mounted catheter is arranged at the distal end of the cable assembly. The ablation device is designed to avoid excessive heating in areas which are not foreseen for the actual treatment. Especially the cable assembly needs to be temperature controlled to avoid damage or an unpleasant temperature level for the operator or the patient. This is due to the fact that during the treatment at least parts of the cable assembly can come into contact with the patient. Cable assemblies known from the prior art typically comprise an envelope which surrounds multiple lines in the form of cables for transferring energy, signaling, control and tubes for cooling. But with the known designs increased power levels lead to undesired or even harmful surface temperatures and especially thermal hot spots around the transferring cable assembly arranged outside of the body of the patient,

The problem with known designs is that the cross-sections of the known cable assemblies are often not rotationally symmetric and may vary over the length. This can lead to non-uniform temperature distributions in the cable assembly. The temperature and its distribution at the outer surface of the cable assembly mainly depends on the transmitted power of the at least one inner conductor of the coaxial cable of the cable assembly and the power losses converted to heat. Other factors which influence the temperature distribution are e.g. the thermal conductivity of the surrounding materials, the ventilation and if used the temperature and flow of the used cooling medium inside of the cable assembly. Although in most cases increasing the cross-section of the inner conductor would allow to reduce the power dissipation and therefore the amount of radiated heat energy, this is not a feasible option as it is at the same time desired to keep the catheter/cable diameter as small and therefore flexible as possible.

For radio frequency ablation, changing the cable impedance has already been proposed. EP 3549544 A1 claims to reduce the power dissipation of a cable assembly with the same diameter by up to 25%. But as stated above already, increasing the diameter of the inner conductor is not a feasible option as a smaller cable diameter with high flexibility is desired, which limits the size of the cooling tubes or layer of dielectric of cable when using the conventional design. Another attempt to reduce the surface temperature is to use a cooling agent either unidirectional with liquid exiting in the human body at the distal end as e.g. proposed in CA 2635316 A1, or using at least two separated channels allowing forward and backward transport of the cooling agent. The cooling agent typically is either a liquid, gas or phase changing material with a boiling temperature between 30° C. to 40° C.,

The problem of the proposed devices known form the prior art is that the proposed designs still cannot keep the surface temperature of the cable assembly between the generator and the handle below an unpleasant or even harmful temperature. In particular, these solutions do not address the problem of hot-spots. A power dissipating wire may come quite close to the outer surface of the cable assembly and may only be separated from sensitive human tissue by a thin polymer layer. Especially when being placed on parts of the human skin with fine blood vessels, the temperature of the cable assembly should preferably not exceed 41° C. As limiting the power for the application is not a feasible option, this usually requests a huge cooling effort and more elaborate design, Introducing a thermal conductive layer as described hereinafter in more detail allows to operate the cable assembly and therefore the overall ablation device with higher power for the treatment, without the risk of hot spots on the outer surface of the cable assembly. Depending on the field of application, arranging a thermal conductive layer within the cable assembly may allow for certain short term treatments even without cooling measures involving the use of expensive and risky cooling agents in the human body.

A cable assembly according to the present disclosure for medical radio frequency or microwave ablation processes, comprises a coaxial cable which comprises an inner conductor, encompassed by a dielectric layer and encompassed by an outer conductor. Typically, standard coaxial cables can be used, which comprise an inner conductor, which can be either solid or stranded and made of a copper-plated steel wire. Between the inner conductor and the outer conductor, a dielectric layer in the form of solid plastic, a foam plastic, or air with spacers supporting the inner conductor can be used. The outer temperature of the cable assembly primarily depends on the power dissipation of the inner conductor within the coaxial cable. For reducing the heat radiation of the coaxial cable, the cable assembly comprises a first cooling line.

Nevertheless, especially in cases where the therapy interval is short, the cooling effect of the first cooling line can be insufficient to avoid thermal peaks at the outer surface of the cable assembly due to the high power levels. Especially thermal peaks at the outer surface of the cable assembly which exceed a temperature of 41° C. can be dangerous. An outer thermal conductive layer encompassing the first cooling line and the coaxial cable can be used to minimize these thermal peaks by dissipating and distributing the thermal energy along the cable perimeter. A solid tube of a material with good thermal conductivity, like copper or silver would be a suitable heat distribution layer for applications where flexibility of the cable bundle or catheter is not required. Nevertheless, for medical treatments like radio frequency or microwave ablation, the flexibility of the cable assembly is crucial. The cable assembly comprises a thermal conductive layer which in a cross-sectional view encompasses the coaxial cable and the first cooling line in a circumferential manner. The thermal conductive layer can thereby be in physical contact with the outer surface of the coaxial cable and the outer surface of the first cooling line and function as a thermal bridge. The thermal conductive layer is configured to especially reduce a thermal peak caused by the coaxial cable by transferring energy, in particular by distributing thermal energy around the coaxial cable and the first cooling line in a circumferential manner.

The thermal conductive layer should be designed to also achieve the desired flexibility. A metal spiral, which is more flexible than a solid tube can be used as it allows bending of the coaxial cable and the first cooling line. Nevertheless, a metal spiral is not very efficient for longitudinal heat transfer. Good results regarding a beneficial circumferential and longitudinal heat transfer can be achieved, when the thermal conductive layer is designed as a braid made from thermally conductive wires and/or is made from a foil, especially a coiled tape. A braid which forms a sleeve encompassing the coaxial cable and at least the first cooling line has proven to be an efficient solution for cooling. A braid arranged under the hose which encompasses the cable assembly is less efficient but sufficient to keep the overall temperature of the cable assembly below the relevant surface temperature.

Good results can be achieved when the thermal conductive layer is mounted by pulling it over the coaxial cable and the first cooling line. In addition, during the assembly of the cable assembly by pulling in required cooling lines and the coaxial cable into the thermal conductive layer, additional wiring can be added to the cable assembly. Alternatively, besides a braid other heat distribution layers with good thermal conductivity, e.g. like corrugated copper foil, structured/perforated coper foil or composite materials like extruded polymer with carbon nanotubes can be used as well. A braid with either flat or round wires provides a heat distribution over the complete outer surface. To keep the cable assembly or catheter sterile and provide a clear separation from the inside to the outside, an additional outer thin polymer cable jacket in the form of a hose can be arranged on the cable assembly or catheter. The high thermal capacity of a metal braid compared to polymer materials in the cable assembly helps to keep the surface temperature well under the harmful level at least for the plurality of comparable short time treatments, usually in the range of a few minutes per treatment.

In a variation the cable assembly may comprise a second cooling line. Typically, the first cooling line is in direct physical contact with the coaxial cable along the main extension direction of the inner conductor and configured to absorb thermal energy of the inner conductor. The inner diameter of the first cooling line can be larger than the inner diameter of the second cooling line. This is especially advantageous when the second cooling line functions as a feeding line and the first cooling line as the actual cooling line, interconnected to the coaxial cable. The varying inner diameter between the first cooling line and the second cooling line allows to influence the flow rate in a beneficial way. A larger inner diameter of the first cooling line leads to a slower flow rate which allows a better heat transfer between the coaxial cable and the cooling medium inside the first cooling line and thereby an improved cooling effect. The second cooling line can be arranged inside the thermal conductive layer. This allows for a particularly dense and therefore space saving design.

Good results can be achieved when a hose is arranged between the thermal conductive layer and the therein arranged coaxial cable and the first cooling line and/or the second cooling line. In a variation, the hose is designed as a shrinking hose which increases the surface pressure between the coaxial cable and the first cooling line and/or the second cooling line, which also improves the heat transfer and therefore the overall cooling effect. After the coaxial cable and the first cooling line and/or the second cooling line are assembled, the shrinking hose is shrunk to achieve a very dense design. Therefore, the thermal conductive layer, the therein arranged coaxial cable and the first cooling line and the second cooling line are arranged in a dense packing with the help of the hose. The dense packing can have a cross-sectional view having an essentially triangular cross section. The cable assemblies are typically produced in small quantities so that the elements can be assembled by pulling the coaxial cable, the first cooling line and optionally the second cooling line and if required additional wiring into the thermal conductive layer. By friction between the thermal conductive layer in form of a braid and the coaxial cable and the first cooling line, the braid tends to contract when pulling the braid over the therein arranged coaxial cable and the first cooling line. Therefore, good results can be achieved when the metal braid is pushed instead. Good results can be achieved when the dense packing comprising the coaxial cable, the first cooling line and optionally the second cooling line and if required additional wiring are assembled with the shrinking hose in a first step, before the metal braid is pushed over the dense packing instead.

By using a prefabricated hose which comprises a thermal conductive layer in form of a metal sheath, the contraction in comparison to the contraction of a metal braid is reduced and so the coaxial cable and cooling lines slide in much easier such that the outer diameter can also be reduced in size, e.g. from 14 mm to 11 mm. The clearance between the coaxial cable, the at least one cooling line and the thermal conductive layer leads to a better flexibility of the overall cable assembly. In another variation the second cooling line can be arranged outside of the thermal conductive layer. This arrangement leads to a better thermal isolation between the second cooling line and the coaxial cable which is together with the first cooling line encompassed by the thermal conductive layer. This allows that the cooling fluid can be guided through the second cooling line without already getting heated up before reaching the distal end of the cable assembly and being returned through the first cooling cable. At least one of the first cooling line and/or the second cooling line are in direct thermal contact with the coaxial cable to provide a heat sink for the coaxial cable,

For medical treatments, especially for radio frequency or microwave ablation, an integral design of the treatment device is desired as it is not feasible to sterilize individual components adequately. Therefore, the transition between the generator and the catheter is preferably designed as an integral ablation device. The ablation device typically comprises a cable assembly as described above in more detail. The ablation device in addition comprises a handle arranged at a distal end of the cable assembly and configured to interconnect to a catheter. For a fast and standardized connection of the ablation device to a generator, a connector assembly is arranged at a proximal end of the cable assembly.

For an efficient assembly, the housing of the handle can be designed as two half-shells which are positioned with respect to each other via pins and connected along a parting plane. Alternatively, or in addition, the two half-shells can also be connected to each other via a circumferential tongue and groove connection and/or be glued or welded together. Good results can be achieved when the thermal conductive layer is interconnected to the housing and acts as a strain relieve means. The thermal conductive layer can be clamped between a collar which is connected to or part of the handle and a sleeve which is mounted onto the collar. When a funnel shaped collar is interconnected to the housing, the thermal conductive layer can be clamped between the funnel shaped collar and a sleeve which is mounted onto the collar. The outer hose can be connected to the handle with or without the thermal conductive layer. For keeping the temperature of the handle and the therein arranged catheter on a level such that the handle can be held by the operator, the housing can encompass a cooling block with cooling channels.

The cooling block can be one-pieced or comprise a connector element and a receiving element for receiving the actual therapeutic tool. The cooling block is typically designed to form a cooling circuit between the first and the second cooling line. In a variation the cooling channels are in fluid connection with at least the first cooling line. The connection between the coaxial cable and the cooling block is typically realized via standard connectors. Good cooling results can be achieved when the cooling channels are arranged in the cooling block encompassing the catheter in a circumferential manner. The catheter itself can comprise at least one cooling line to also keep the temperature of the catheter below the critical temperature of 41° C. To enable that the cooling medium of the cable assembly can also be used to cool the catheter, a cooling circuit needs to be established. Therefore, the cooling block can comprise a receiving space for receiving a holder for the catheter which holder forms a cooling channel with the cooling block in a mounted position. In a preferred design the holder is designed as a rotationally symmetrical, essentially cylindrical element which is connected to the cooling block via a plug-in connection. In the mounted state the back wall of the receiving space and the holder form a groove which can be flushed with the cooling medium. In a preferred variation, the cooling medium is guided through the first cooling line from the connector element into the cooling block and through the catheter, preferably to a tip of the catheter, and is returned through the catheter to the cooling block. From the cooling block, the cooling medium is returned through the second cooling line back to the connector element to close the circuit. Alternatively, the cooling circuit can be established reversed.

For a fast and efficient connection of the cable assembly and the overall ablation device, the cable assembly typically comprises a connector assembly. The connector assembly is configured to be used in the ablation device. The connector assembly comprises a housing with a therein arranged electrical connector for connecting the coaxial cable to the generator and a breakout section with a pigtail for connecting at least the first cooling line to the cooling device. The connector assembly is typically arranged at the proximal end of the cable assembly for interconnecting the ablation device to a generator. To be able to set up a cooling circuit, the breakout section can extend away from the connector assembly to deflect the cable assembly and the pigtail. The pigtail can comprise at least one squeezable connection line. The at least one connection line can be split in two lines merging into a hollow needle. The section of the pigtail between the breakout section of the connector assembly and the needle can be clamped into a pump. Good results can be achieved when the cooling medium is extracted through the needle out of a reservoir of cooling medium and pumped through the pigtail and the second cooling line to the handle before it returns through the first cooling line, closing the cooling circuit. Therefore, the squeezable connection line of the pigtail can merge into a hollow needle. For hygiene reasons infusion containers with saline solution can be used as a reservoir of cooling medium. In addition to the electrical connector, the connector assembly can contain additional connectors for additional wiring, e.g. for a thermocouple, control lamp etc. The ablation device typically comprises a connector assembly, as herein described, which can comprise an electrical connector and a mating electrical connector, as herein described.

For being able to connect both, the coaxial cable as well as additional wiring, e.g. for a thermocouple, control lamp etc., typically an electrical connector with a coaxial connector and additional electrical contacts is used. In a preferred variation, the electrical connector comprises a base element made form an insulating material. The base element can comprise a front part and a rear part, with at least two electrical contacts being arranged in the front part of the base element. The front part of the base element can be the main body of the overall electrical connector. The front part can be a rotational symmetrical part, e.g. in the form of a sleeve, Typically, both the electrical contacts as well as the coaxial connector for transmitting e.g. the radio frequency or microwave, can be arranged in the base element. Alternatively, the at least two electrical contacts can also be arranged in a separate holder, which typically encompasses the base element.

The coaxial connector can be in the form of a standardized coaxial connector, comprising an inner contact and an outer contact spaced apart from each other. The coaxial connector can be arranged in the base element, with the outer contact of the coaxial connector being preferably arranged in a bore of the base element and being encompassed by the base element. Good results can be achieved when the coaxial connector is a standard radio frequency connector, which is modular regarding the cable entry to cover different cable sizes according to customer needs. The inner contact of the coaxial connector can be in form of a center pin, which can comprise at a first end flexible tongues for receiving an inner contact of the mating electrical connector in the mounted position. The second end can comprise a receiving space for a cable end sleeve. The inner contact is typically arranged in the outer contact and spaced apart by at least one insulating element.

The coaxial connector is typically spring mounted along a center axis of the electrical connector with respect to the base element. Good results can be achieved, when the coaxial connector comprises an outer contact, which is interconnected to the base element by a spring. During operation, the spring ensures that even under changing loads, e.g. cyclical loads or vibrations, the coaxial connector remains interconnected to a mating coaxial connector. In an unconnected state, a front end of the coaxial connector typically protrudes along a center axis of the electrical connector beyond the front end of at least one of the at least two electrical contacts. This ensures that during mating of the electrical connector with a mating electrical connector, the coaxial cable is connected before the at least two electrical contacts are mated.

In addition, the at least two electrical contacts are preferably arranged in a circumferentially manner with respect to the coaxial connector. The front part of the base element is typically the carrier of the coaxial connector for connecting the radio frequency line or microwave line and for the at least two electrical contacts for connecting direct current (DC) lines. The number of electrical contacts for the DC lines can vary, typically the number of electrical contacts is within a range from 2-12 contacts. The electrical contacts can be arranged circumferential with respect to the coaxial connector, being rotationally symmetrical with respect to the center axis. To ensure that the electrical connector is properly connected to mating connector, the at least two electrical contacts can be each at least partially spring mounted with respect to the base element, as a safety measure. The springs ensure that the electrical contacts will be separated by the spring force when the electrical connector is not fully inserted and latched. When the electrical contacts are not in electrical contact, a control unit can detect the missing signal and assure that the coaxial connector will not be powered.

Good results can be achieved, when each of the at least two electrical contacts comprise a front part and a rear part which are interconnected to each other by a spring. The front part can be a rotational symmetrical pin, which can comprise a contact surface which has a spherical shape. The contact surface is typically arranged at the front end of the electrical connector and configured for electrically connecting the electrical contact to an electrical contact of a mating electrical connector. The front part of the at least two electrical contacts can each comprise at a rear end a funnel shaped protrusion which is in the mounted state encompassed by the spring. The protrusion is configured to at least partially plunge into a receiving space of the rear part of the respective electrical contact in the mounted state. The front part is typically arranged in a mounted state in a bore of the base element. To prevent that, the front part is pushed out of the base element in an unwanted manner by the spring. The front part can comprise a collar which abuts against a stop in the base element. The stop is configured to limit the axial movement of the electrical contact with respect to the base element towards a front end of the electrical connector.

The rear part of the electrical contact typically comprises at a front end flexible tongues, which function as a receiving space for the rear end of the front part. The rear part can comprise in the axial directional a first and a second collar. The first collar can function as a spacer, which in the mounted position spaces the rear part of the base element at a distance from the front part of the base element. The second collar can function as a locking means, which in the mounted position is pressed together with the base element. This can ensure that the movement of the electrical contact is limited in axial direction with respect to a rear end of the electrical connector, Each of the at least two electrical contacts may in addition comprise an adapter part. The adapter part is typically configured for interconnecting the electrical contacts to a conductor or an electronic component. Depending on individual customer needs, the connection may be a straight or a right-angled configuration for wires, a PCB, etc. The adapter part can comprise a receiving space for a conductor which is interconnectable to the adapter part, preferably by a crimp or soldering connection. Alternatively, the adapter part can comprise a contact pin for connecting the adapter part to an electronic component, preferably a printed circuit board, in particular by a soldering connection. In the mounted position, the adapter part is typically connected to the rear end of an electrical contact, preferably by a plug connection.

For interconnecting the electrical connector to a mating connector, the electrical connector may comprise a plug element, which plug element typically encompasses a locking element. The locking element can be a deformable locking ring, which is arranged in a at least partially circumferential groove of the plug element. Typically, the locking ring is a C-shaped ring which during interconnection is widened in circumferential direction and in the mounted state engages with a connection element of the mating electrical connector, e.g. a collar. When being pushed over the connection element, the locking ring typically widens up and snaps into a recess formed between the connection element and the base element of the mating electrical connector and thereby secures the electrical contact in axial direction.

Good results can be achieved when the deformable locking ring comprises a locking surface which with respect to the center axis is inclined by an angle of 35°-42°, preferably 36°-38°, most preferably 38°. It has shown that in particular an angle of essentially 38° proofs to be a well-balanced compromise between an appropriate holding force to prevent an unwanted disengagement of electrical connector and mating electrical connector and a pleasant operability for the operator. To prevent an abrasion of the mating electrical, polymer solutions, e.g. Polyether ether ketone (PEEK) or Polyimide (PI) for the locking ring and Polyoxymethylene (POM) for the electrical connector, have proven a suitable material combination. In addition, a polymer based electrical connector reduces the overall weight. The unlocking element can be in form of an unlocking ring, which comprises an unlocking shoulder which during unlocking engages with the locking surface and widens the locking ring.

A mating electrical connector for interconnection with the electrical connector, typically comprises, a coaxial connector for transmitting a radio frequency or microwave, comprising an inner contact and an outer contact spaced apart from each other and being arranged in a base element. The coaxial connector of the mating electrical connector is interconnectable to the coaxial connector of the electrical connector, it typically protrudes above the at least two electrical connectors of the mating electrical connector. This design ensures that the coaxial connector connects first with the counterpart before the electrical contacts are connecting. This assures a controlled start up. While disconnecting vice versa, the preferably direct current contacts disconnect before the coaxial connector is disconnected,

In a preferred variation, the coaxial connector and/or the at least two electrical contacts of the mating electrical connector are designed as female connectors. The mating electrical connector typically also comprises at least two electrical contacts being arranged in a circumferential manner with respect to the coaxial connector. The at least two electrical contacts can comprise a bore or otherwise a concave contact surface, to improve the performance even under vibrations and to additionally guide the pins when mating. Good results regarding electrical contact are achieved, when the contact surface of the at least two electrical contacts of the electrical connector are convex, especially spherical, and the contact surfaces of the electrical contacts of the mating electrical connector comprise a bore or are otherwise concave, that the convex spherical contact surface makes a ring shaped contact when mated. Respectively, the electrical contacts of the electrical connector and the electrical contacts of the mating electrical connector can be vice versa. The mating electrical connector can comprise a connection element which comprises a positioning element configured to align at least two electrical contacts of the mating electrical connector with the at least two electrical contacts of the electrical connector during mating. The mating coaxial connector can in addition comprise a positioning element which is configured to align the at least two electrical contacts of the mating electrical connector. A number of positioning elements can be arranged at the base element, e.g. in the form of pins, to avoid that erroneously unsuitable devices are connected to the mating electrical connector. Alternatively, or in addition, the electrical connector can comprise at least one guiding element and/or a thereto respective counterpart, e.g. a groove or recess. The inner contact of the coaxial connector of the mating electrical connector can comprise a replaceable tip. The inner contact of the mating coaxial connector can be made in two parts, with an abrasion resistant tip, e.g. stainless steel like XCrNiCuS18-9-2 to guarantee a number of matings being greater than 10′000.

It is to be understood that both the foregoing general description and the following detailed description present embodiments, and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF SEVERAL VIEWS OF THE DRAWINGS

The herein described disclosure will be more fully understood from the detailed description given herein below and the accompanying drawings which should not be considered limiting to the disclosure described in the appended claims.

FIG. 1 shows a schematic cross sectional view of a first variation of the cable assembly;

FIG. 2 shows a schematic cross sectional view of a second variation of the cable assembly;

FIG. 3 shows a schematic cross sectional view of a third variation of the cable assembly;

FIG. 4 shows a perspective lateral view with a partial cut-out of a first variation of the handle of the ablation device;

FIG. 5 shows a schematic top view with a partial cut-out of the first variation of the handle of the ablation device according to FIG. 4;

FIG. 6 shows a lateral view with a partial cut-out of a variation of the connector assembly;

FIG. 7 shows a perspective view from above and the front with a partial cut-out of the variation of the connector assembly according to FIG. 6 and thereto connectable mating electrical connector;

FIG. 8 shows a perspective view from above and the front with a partial cut-out of the variation of the mating electrical connector according to FIG. 7;

FIG. 9 shows a perspective exploded view from above and behind on the electrical connector according to FIG. 6;

FIG. 10 shows a detailed perspective exploded view from above and behind on the electrical connector according to FIG. 9;

FIG. 11a shows a perspective view from above and behind on a first variation of the rear part of the electrical contact;

FIG. 11b shows a sectional view from above and behind on a first variation of the rear part of the electrical contact;

FIG. 12a shows a perspective view from above and behind on a second variation of the rear part of the electrical contact; and

FIG. 12b shows a sectional view from above and behind on a second variation of the rear part of the electrical contact.

### DETAILED DESCRIPTION OF THE INVENTION

Reference will now be made in detail to certain embodiments, examples of which are illustrated in the accompanying drawings, in which some, but not all, features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

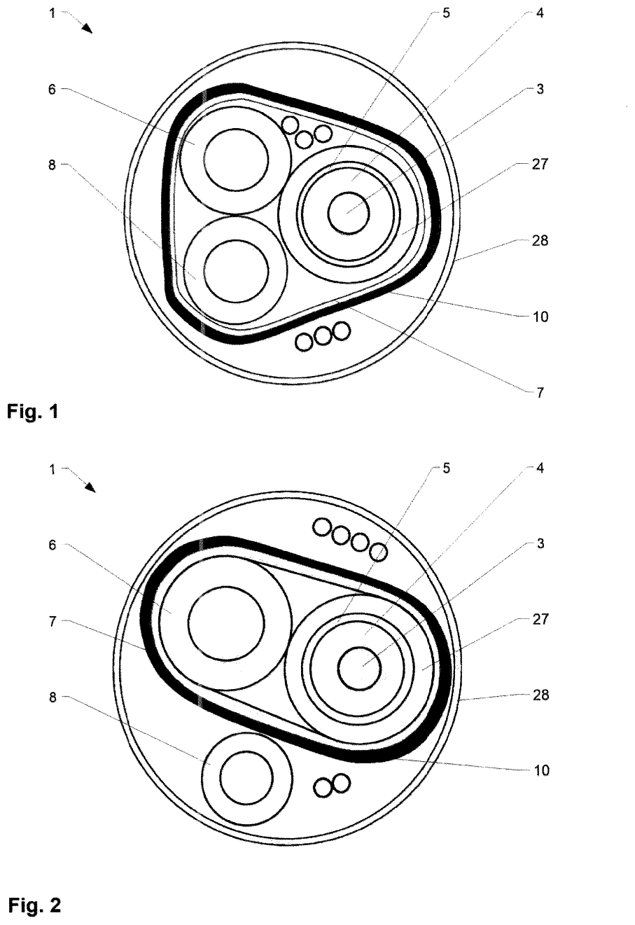

As can be obtained best from FIGS. 1 to 3, all three variations of the cable assembly 1 for medical radio frequency or microwave ablation comprise a coaxial cable 2, which comprises an inner conductor 3, encompassed by a dielectric layer 4, encompassed by an outer conductor S. In addition, all variations comprise a first cooling line 6 which is in physical contact with the coaxial cable. The shown thermal conductive layer 7 in a cross-sectional view encompasses the coaxial cable 2 and the first cooling line 6 in a circumferential manner and is configured to reduce a thermal peak caused by the coaxial cable 2 when transferring energy, by distributing thermal energy around the coaxial cable 2 and the first cooling line 6 in a circumferential manner. The shown thermal conductive layers 7 are in physical contact with the outer surface of the coaxial cable 2 and the outer surface of the first cooling line 6 and function as a thermal bridge. In addition, all variations also comprise a second cooling line 8.

FIG. 1 shows a schematic cross sectional view of the first variation of the cable assembly 1. In the shown variation the first cooling line 6 and the second cooling line 8 are in direct thermal contact with the coaxial cable 2 to provide a heat sink for the coaxial cable 2. In addition, the second cooling line 8 is also arranged inside the thermal conductive layer 7. The thermal conductive layer 7, the therein arranged coaxial cable 2 and the first cooling line 6 and the second cooling line 8 in a cross-sectional view have an essentially triangular cross section. The dense design is achieved by a hose 10, in the shown variation as a shrinking hose which is arranged between the thermal conductive layer 7 and the therein arranged coaxial cable 2 and the first 6 and the second 8 cooling line. The shrinking hose 10 increases the surface pressure between the coaxial cable 2 and the first cooling line 6 and the second cooling line 8, which also improves the heat transfer and cooling effect. This design allows for a particular dense and therefore space saving design. The shown cable assembly 1 is produced in small quantities so that the elements can be assembled by pulling the coaxial cable 2, the first cooling line 6 and optionally additional wiring into the thermal conductive layer 7. By friction between the thermal conductive layer 7 in form of a braid 9 and the coaxial cable 2 and the first cooling line 6, the braid 9 tends to contract when pulling the braid 9 over the therein arranged coaxial cable 2 and the first cooling line 6. Alternatively, other thermal conductive layers 7 with good thermal conductivity, e.g., like corrugated copper foil, structured/perforated coper foil or composite materials like extruded polymer with carbon nanotubes can be used as well.

FIG. 2 shows a schematic cross sectional view of a second variation of the cable assembly 1. In the shown variation the second cooling line 8 is arranged outside of the thermal conductive layer 7. In addition, the inner diameter of the first cooling line 6 is larger than the inner diameter of the second cooling line 8. This is especially advantageous when the second cooling line 8 functions as a feeding line and the first cooling line 6 as the actual cooling line, interconnected to the coaxial cable 2. The varying inner diameter between the first cooling line 6 and the second cooling line 8 allows to influence the flow rate in a beneficial way. A larger inner diameter of the first cooling line 6 leads to a slower flow rate which allows a better heat transfer between the coaxial cable 2 and the cooling medium inside the first cooling line 6 and thereby an improved cooling effect. This design leads to a better thermal isolation between the second cooling line 8 and the coaxial cable 2 which is together with the first cooling line 6 encompassed by the thermal conductive layer 7. This allows that the cooling fluid can be guided through the second cooling line 8 without already getting heated up before reaching the distal end 12 of the cable assembly 1 and being returned through the first cooling line 6. In the shown variation only the first cooling line 6 is in direct thermal contact with the coaxial cable 2 to provide a heat sink for the coaxial cable 2.

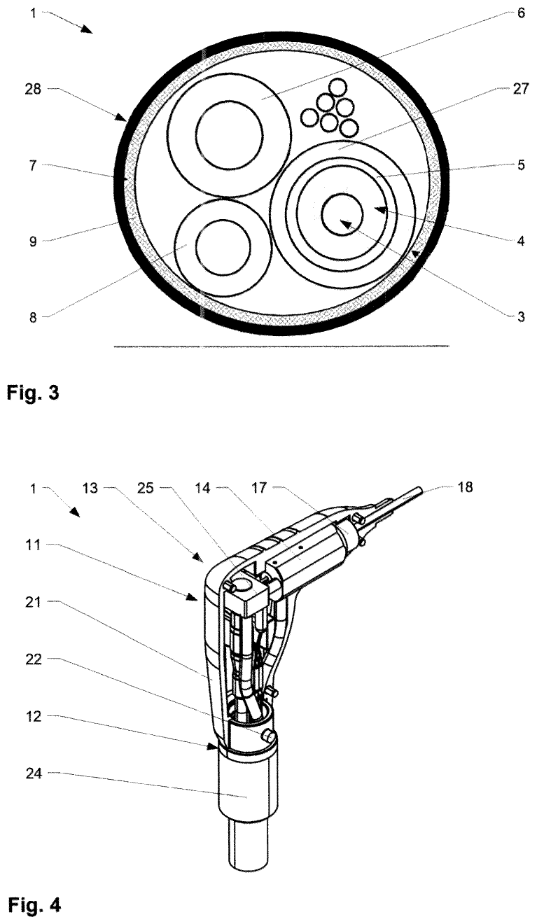

FIG. 3 shows a schematic cross sectional view of a third variation of the cable assembly 1. The shown thermal conductive layer 7 comprises a braid 9 which is made from thermally conductive wires and/or is a foil. The inner diameter of the first cooling line 6 is again larger than the inner diameter of the second cooling line 8. As metal spirals which are used for mechanical purposes as they prevent the compression/squeezing of the first 6 and the second 8 cooling lines, are not very efficient for longitudinal heat transfer, a braid 9 as shown is used. A metal braid 9 leads to good results regarding the circumferential and longitudinal heat transfer. The braid 9 is preferably made from thermally conductive wires and/or made from a foil. A braid 9 sleeve has proven to be an efficient solution for cooling. A braid 9 arranged under the outer hose 28 of the cable assembly 1 is less efficient but sufficient to keep the overall temperature of the cable assembly 1 below the relevant surface temperature.

The first 6 and/or the second 8 cooling lines are pulled in in a pre-determined sequence or all together with the coaxial cable 2 into the braid 9, as well as additional wiring if required. A braid 9 with either flat or round wires provides a beneficial heat distribution over the complete outer surface of the cable assembly. To keep the cable assembly 1 or catheter sterile and provide a clear separation from the inside to the outside, an additional outer thin polymer cable jacket 27 can be arranged on the cable assembly 2 or catheter. This outer hose 28 encompasses the therein arranged coaxial cable 2, thermal conductive layer 7 and the first 6 and the second 8 cooling line. The high thermal capacity of a metal braid 9 compared to polymer materials in the cable assembly 1 helps to keep the surface temperature well under the harmful level at least for the plurality of comparable short time treatments, usually in the range of a few minutes per treatment. By using a prefabricated hose 28 which comprises a thermal conductive layer 7 in form of a metal sheath, the contraction in comparison to a braid 9 is reduced and so the coaxial cable 2 and cooling lines 6,8 slide in much easier so that the outer diameter can also be reduced in size. E.g from 14 mm to 11 mm. The clearance between coaxial cable, 2, cooling lines 6, 8 and the braid 9 leads to a better flexibility of the overall cable assembly 1.

FIG. 4 shows a perspective lateral view with a partial cut-out of a first variation of the handle 11 of the ablation device 29. The shown handle 11 is arranged at the distal end 12 of the cable assembly 1 and comprises a housing 13 which encompasses the cooling block 14 with therein arranged cooling channels 15. The shown integral design of the ablation device 29 is designed to avoid that individual components have to be sterilized individually. Therefore, the shown ablation device 29 is realized as a single pieced assembly for single use. For an efficient assembly, the shown housing 13 is designed as two half-shells 21 which are positioned with respect to each other via pins 22 and connected along a parting plane. Good results can be achieved when the thermal conductive layer 7 is interconnected to the housing 13 and acts as a strain relief means. In the shown variation the thermal conductive layer 7 is clamped between a collar 23 which is connected to the handle 11. Alternatively, the collar 23 can also be part of the handle 11. The shown sleeve 24 is mounted onto the collar 23 and fixates the thermal conductive layer 7. The outer hose 28 can be connected to the handle 11 with or without the thermal conductive layer 7. Especially with the first or the second variation of the cable assembly 1 it is difficult to keep the thermal conductive layer 7 attached solely by clamping. Therefore, the thermal conductive layer 7 can be additionally glued or fusion welded to the handle 11.

As can be best obtained from FIG. 4, the cooling block 14 can be one-pieced or as shown comprise a connector element 25 and a receiving element 26 for receiving the actual therapeutic tool. The cooling block 14 is designed to form a cooling circuit between the first cooling line 6 and the second cooling line 8. In a variation the cooling channels 15 are in fluid connection with at least the first cooling line 6. The connection between the coaxial cable 2 and the cooling block 14 is in the shown variation realized via a standard connector. To realize the cable assembly 1 as an integral unit which is easy to change, the cable assembly 1 can comprise a connector assembly 19 which is arranged at a proximal end of the cable assembly 1.

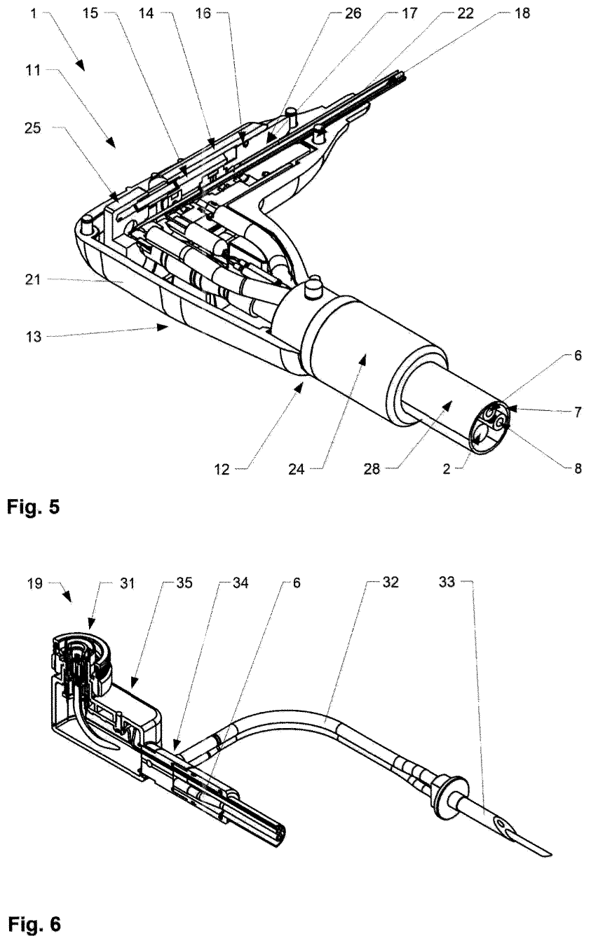

FIG. 5 shows a schematic top view with a partial cut-out of the first variation of the handle 11 of the ablation device 29. The connector element 25 and the receiving element 26 for receiving the catheter 18 are connected to each other with pins which at the same time function as cooling lines forming a cooling circuit between the connector element 25 and the receiving element 26. In the shown variation the cooling channels 15 are in fluid connection with at least the first cooling line 6 and the dielectric layer 5 is interconnected to the cooling block 14. The shown cooling channels 15 are arranged in the cooling block 14 encompassing the catheter 18 in a circumferential manner. The shown catheter 18 itself also comprises cooling lines to also keep the temperature of the catheter 18 below the critical temperature of 41° C. To enable that the cooling medium of the cable assembly 1 can also be used to cool the catheter 18, a cooling circuit needs to be established. Therefore, the shown cooling block 14 comprises a receiving space 16 for receiving a holder 17 for the catheter 18 which holder 17 forms a cooling channel 15 with the cooling block 14 in a mounted position. In the shown design the holder 17 is designed as a rotationally symmetrical, essentially cylindrical element which is connected to the cooling block 14 via a plug-in connection. In the mounted state the back wall of the receiving space 16 and the holder 17 form a groove which can be flushed with the cooling medium.

FIG. 6 shows a lateral view with a partial cut-out of a variation of the connector assembly 19. The shown connector assembly, arranged at the proximal end of the cable assembly 1 for interconnecting the ablation device 29 to a generator and a cooling device, comprises an electrical connector 31 for connecting the coaxial cable 2 to the generator. The connector assembly 19 comprises a breakout section 34, designed as a cylindrical nozzle, which extends away from the connector assembly 19, to deflect the cable assembly 1 and the pigtail 32. As can be obtained from FIG. 6, the pigtail 32 for connecting the first cooling line 6 to the cooling device merges into a hollow needle 33. The section of the pigtail between the breakout section 34 of the connector assembly 19 and the hollow needle 33 can be clamped into a pump. The cooling medium is thereby extracted through the needle 33 out of a reservoir of cooling medium and pumped through the pigtail 32 and the second cooling line 8 to the handle 11 before it returns through the first cooling line 6, closing the cooling circuit.

The shown connector assembly 19 arranged at the proximal end 20 of the cable assembly 1 comprises an electrical connector 31 for connecting the inner conductor 3 of the coaxial cable 2 to a generator. The electrical connector 31 comprises a coaxial connector and can be connected to a mating connector 19 arranged at the generator. The electrical connector 31 is arranged at the connector assembly 19 which comprises assembly space for additional power electronics. The shown connector assembly 19 further comprises a connection line 32 for connecting the first cooling line 6 to a reservoir of cooling medium. For hygiene reasons infusion containers with saline solution can be used as a reservoir of cooling medium. For obtaining a fast connection the connection line 32 of the shown variation merges into a hollow needle 33 which is configured to be pierced into the reservoir of cooling medium.

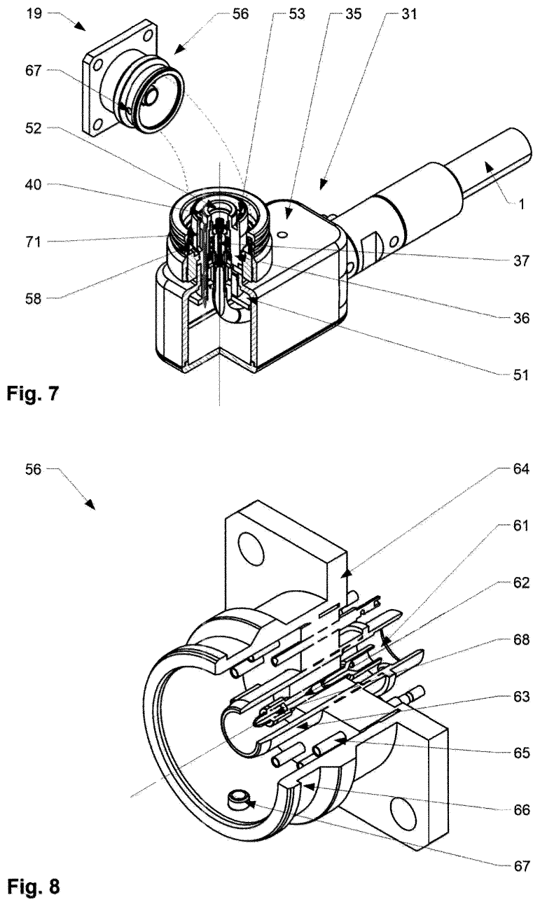

FIG. 7 shows a perspective view from above and the front with a partial cut-out of the first variation of the connector assembly 19 and a thereto connected mating electrical connector 56. For being able to connect both, the coaxial cable as well as additional wiring, e.g. for a thermocouple, control lamp etc., the shown electrical connector 31 comprises a coaxial connector 37 and additional electrical contacts 40. In the shown variation, the electrical connector 31 comprises a base element 36 made form an insulating material. Typically, both the electrical contacts 40 as well as the coaxial connector 37 for transmitting a radio frequency or microwave, are arranged in the base element 36. Alternatively, the at least two electrical contacts 40 can also be arranged in a separate holder, which typically encompasses the base element 36.

FIG. 8 shows a perspective view from above and the front with a partial cut-out of the mating electrical connector 56. The shown mating electrical connector 56 for interconnection with the electrical connector 31 comprises a coaxial connector 61 for transmitting a radio frequency or microwave, which comprises an inner contact 62 and an outer contact 63 spaced apart from each other and being arranged in the base element 64. The coaxial connector 61 of the mating electrical connector 56 is interconnectable to the coaxial connector 37 of the electrical connector 31, it protrudes above the at least two electrical connectors 65 of the mating electrical connector 56. This design ensures that the coaxial connector 61 connects first with the counterpart before the electrical contacts 65 are connecting. While disconnecting, the preferably direct current contacts disconnect before the coaxial connector 61 is disconnected. This ensures that the radio frequency or microwave is immediately shut down, when the electrical connector 31 is disconnected. It is thus avoided that radiation exits the mating electrical connector 56 in an uncontrolled manner.

In the shown variation, the coaxial connector 61 and/or the at least two electrical contacts 65 of the mating electrical connector 56 are designed as female connectors. The mating electrical connector 56 also comprises 12 electrical contacts 65 being arranged in a circumferential manner with respect to the coaxial connector 61. The electrical contacts 65 comprise a groove 70, in the shown variation a spherically shaped groove 70, to increase the contact surface in order to improve the performance even under vibrations and to additionally guide the pins when mating.

The mating electrical connector 56 comprises a connection element 66 which comprises a positioning element 67 configured to align at least two electrical contacts 65 of the mating electrical connector 56 with the at least two electrical contacts 40 of the electrical connector 31 during mating. The mating coaxial connector 56 in addition comprises a positioning element 67 which is configured to align the at least two electrical contacts 65 of the mating electrical connector 56. In addition, the shown electrical connector 31 comprises a counterpart in form of a recess which corresponds to the at least one positioning element 67. The inner contact 62 of the coaxial connector 61 of the mating electrical connector 56 comprise a replaceable tip 68. The inner contact 62 of the mating coaxial connector 56 is made in two parts, with an abrasion resistant tip 68, e.g. made from stainless steel.

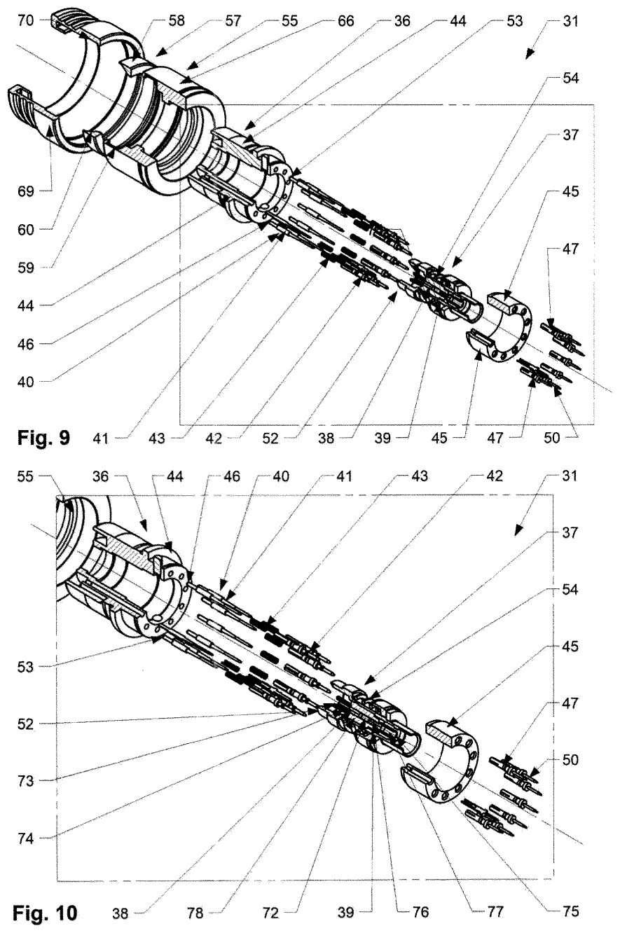

FIG. 9 shows a perspective exploded view from above and behind on the electrical connector 31. The shown coaxial connector 37 is in form of a standardized coaxial connector 37, comprising an inner contact 38 and an outer contact 39 spaced apart from each other. The coaxial connector 37 is arranged in the base element 36, with the outer contact 39 of the coaxial connector 37 being arranged in a bore of the base element 36 and being encompassed by the base element 36. The shown base element 36 comprises a front part 44 and a rear part 45, with at least two electrical contacts 40 being arranged in the front part 44 of the base element 36. The front part 44 of the base element 36 is the main body of the overall electrical connector 31. The shown front part 44 is a rotational symmetrical part, in the shown variation in form of a sleeve,

The shown coaxial connector 37 is a standard radio frequency connector, which is modular regarding the cable entry to cover different cable sizes according to customer needs. The inner contact 38 of the shown coaxial connector 37 is in form of a center pin 72 which comprises at a first end 73 flexible tongues 74 for receiving the inner contact 62 of the mating electrical connector 56 in the mounted position. The second end 75 comprises a receiving space 76 for a cable end sleeve 77. The shown inner contact 62 is arranged in the outer contact 63 and spaced apart by at least one insulating element 78.

The coaxial connector 37 of the shown variation is spring mounted along a center axis of the electrical connector 19 with respect to the base element 36. The coaxial connector 37 comprises an outer contact 39, which is interconnected to the base element 36 by a spring 43. During operation, the spring 43 ensures that even under changing loads, e.g. cyclical loads or vibrations, the coaxial connector 37 remains interconnected to a mating coaxial connector 56. In an unconnected state, the front end of the coaxial connector 37 protrudes along a center axis of the electrical connector 31 beyond the front end of at least one of the at least two electrical contacts 40. This ensures that during mating of the electrical connector 31 with a mating electrical connector 56, the coaxial cable 2 is connected before the at least two electrical contacts 40 are mated.

In addition, the at least two electrical contacts 40 are arranged in a circumferential manner with respect to the coaxial connector 37. The shown front part 44 of the base element 36 is the carrier of the coaxial connector 37 for connecting the radio frequency line or microwave line and for the at least two electrical contacts 40 for connecting direct current (DC) lines. The number of electrical contacts 40 for the DC lines can vary, typically the number of electrical contacts 40 is within a range from 2-12 contacts, with 10 in the shown variation. The electrical contacts 40 are arranged circumferential with respect to the coaxial connector 37, being rotationally symmetrical with respect to the center axis. To ensure that the electrical connector 31 is properly connected to mating connector 56, the at least two electrical contacts 40 are each at least partially spring mounted with respect to the base element 36, as a safety measure for proper contacting. The springs 43 ensure that the electrical contacts 40 of the electrical connector 31 will be separated by the spring force, when the electrical connector 31 is not fully inserted and latched with the mating electrical connector 56. When the electrical contacts 43 of the electrical connector 31 are not in electrical contact with the electrical contacts 65 of the mating electrical connector 56, a control 25 unit can detect the missing signal and assure that the coaxial connector 61 will not be powered.

For interconnecting the electrical connector 31 to a mating electrical connector 56, the shown electrical connector 31 comprises a plug element 55, which plug element 55 encompasses a locking element 57. The locking element 57 of the shown variation is a deformable locking ring 58, which is arranged in a at least partially circumferential groove 59 of the plug element 55. The shown locking ring 58 is a C-shaped ring, which during interconnection is widened in circumferential direction and in the mounted state engages with a connection element 66 of the mating electrical connector 56, e.g. in form of a collar. When being pushed over the connection element 66, the shown locking ring 58 widens up and snaps into a recess formed between the connection element 66 and the base element 64 of the mating electrical connector 56 and thereby secures the at least two electrical contacts 40 in axial direction.

The shown locking ring 58 comprises a locking surface 60 which with respect to the center axis (x) can be inclined by an angle of 35°-42°, preferably 36°-38°, in the shown variation it is inclined by 38°. It has shown that in particular an angle of essentially 38° proofs to be a well-balanced compromise between an appropriate holding force to prevent an unwanted disengagement of electrical connector 31 and mating electrical connector 56 and a pleasant operability for the operator. To prevent an abrasion of the mating electrical connector 56, polymer solutions e.g. in the shown variation an electrical connector 31 made form PI or PEEK for the locking ring 58 and POM for an unlocking element 69 have proven a suitable material combination. The shown unlocking element 69 is in form of an unlocking ring, which comprises an unlocking shoulder which during unlocking engages with the locking surface 60 and widens the locking ring.

FIG. 10 shows a detailed perspective exploded view from above and behind on the electrical connector 31. Each of the shown electrical contacts 40 comprises a front part 41 and a rear part 42. The shown front parts 41 are rotational symmetrical pins, which comprise a contact surface 46 which has a spherical shape. The contact surface 46 is arranged at a front end and configured for electrically connecting the electrical contact 40 to an electrical contact 65 of a mating electrical connector 56. The at least two electrical contacts 65 each comprise at a rear end a funnel shaped protrusion which is in the mounted state encompassed by the spring 43. The protrusion is configured to at least partially plunge into a receiving space of the rear part 42 in the mounted state and thereby make the electrical contact. The front part 41 is arranged in a mounted state in a bore of the base element 36. To prevent that the front part 41 is pushed out of the base element 36 in an unwanted manner by the spring 43. As shown, the front part 41 comprises a collar, which abuts against a stop in the base element 36. The stop is configured to limit the axial movement of the electrical contact 31 with respect to the base element 36 towards a front end of the electrical connector 31.

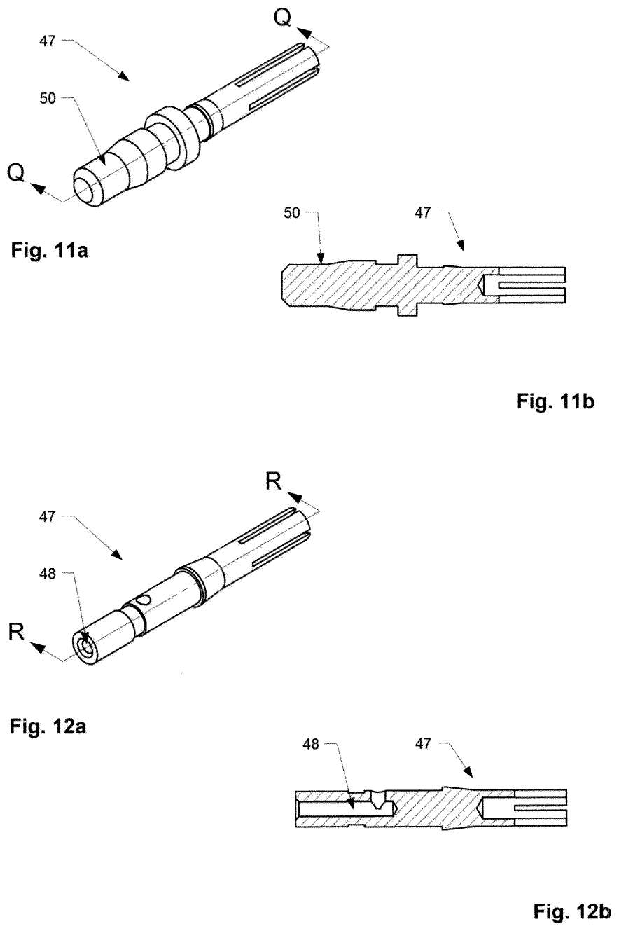

The rear part 42 of the shown electrical contacts 40 typically comprises at the respective front end flexible tongues, which function as a receiving space for the rear end of the front part 41. The rear parts 42 comprise the axial directional a first and a second collar. The shown first collars function as a spacer, which in the mounted position spaces the rear part 45 of the base element 36 at a distance from the front part 44 of the base element 36. The second collar of the shown variation functions as a locking means, which in the mounted position is pressed together with the base element 36. This ensures that the movement of the electrical contact 40 is limited in axial direction with respect to a rear end of the electrical connector 31. Each of the at least two electrical contacts 40 in addition comprises an adapter part 47, shown in FIGS. 11 and 12 in greater detail. The shown adapter part 47 is typically configured for interconnecting the electrical contacts 40 to a conductor or an electronic component. Depending on individual customer needs, the connection may be a straight or a right-angled configuration for wires, a PCB, etc.

FIG. 11 shows a perspective view from above and behind on a first variation of the adapter part 47 of the electrical contact 31 in FIG. 11a and a sectional view thereof in FIG. 11b. The adapter part 47 of the first variation comprises a contact pin for connecting the adapter part to an electronic component, preferably a printed circuit board, in particular by a soldering connection. In the mounted position, the adapter part 47 is typically connected to the rear end of an electrical contact 31, preferably by a plug connection. FIG. 12 shows a perspective view from above and behind on a second variation of the adapter part 47 of the electrical contact 31 in FIG. 12a and a sectional view thereof in FIG. 12b. The adapter part 47 of the second variation comprises a receiving space 48 for a conductor which is interconnectable to the adapter part 47, preferably by a crimp or soldering connection.

Rather, the words used in the specification are words of description rather than limitation, and it is understood that various changes may be made without departing from the Spirit and scope of the disclosure.

1. A cable assembly for medical radio frequency or microwave ablation comprising:
a coaxial cable comprising an inner conductor, encompassed by a dielectric layer, encompassed by an outer conductor;
b, a first cooling line;
c. a second cooling line:
d. a thermal conductive layer in a cross-sectional view encompassing the coaxial cable and the first cooling line in a circumferential manner, configured to reduce a thermal peak caused by the coaxial cable when transferring energy, by distributing thermal energy around the coaxial cable and the first cooling line in a circumferential manner.

2. (canceled)

3. The cable assembly according to claim 1, wherein at least one selected from the group consisting of the first cooling line and the second cooling line are in direct thermal contact with the coaxial cable to provide a heat sink for the coaxial cable.

4. The cable assembly according to claim 1, wherein the thermal conductive layer, the therein arranged coaxial cable and the first cooling line and the second cooling line in a cross-sectional view have an essentially triangular cross section.

5. The cable assembly according to claim 1, wherein the second cooling line is arranged inside the thermal conductive layer.

6. The cable assembly according to claim 1, wherein the second cooling line is arranged outside of the thermal conductive layer.

7. The cable assembly according to claim 1, wherein the thermal conductive layer comprises a braid made from thermally conductive wires or is a foil.

8. The cable assembly according to claim 1, wherein the inner diameter of the first cooling line is larger than the inner diameter of the second cooling line.

9. The cable assembly according to claim 1, wherein a hose is arranged between the thermal conductive layer and the therein arranged coaxial cable and the first cooling line.

10. An ablation device comprising
a. a cable assembly according to claim 1,
b. a handle arranged at a distal end of the cable assembly and configured to interconnect to a catheter, and
c. a connector assembly arranged at a proximal end of the cable assembly.

11. The ablation device according to claim 10, wherein the handle comprises a housing which encompasses a cooling block with at least one cooling channel which is in fluid connection with at least the first cooling line.

12. The ablation device according to claim 11, wherein the cooling block comprises a mounting bore for receiving a holder for the catheter which holder forms a cooling channel with the cooling block in the mounted position.

13. The ablation device according to claim 11, wherein the cooling channels are arranged in the cooling block encompassing the catheter in a circumferential manner in the mounted position.

14. The ablation device according to claim 11, wherein the outer conductor of the coaxial cable is interconnected to the cooling block.

15. The ablation device according to claim 11, wherein the thermal conductive layer is interconnected to the housing, which thermal conductive layer acts as a strain relieve means in the cable assembly.

16. A connector assembly configured to be used in the ablation device according to claim 10, comprising
a. housing with a therein arranged electrical connector for connecting the coaxial cable to the generator and
b. a breakout section with a pigtail for connecting at least the first cooling line to the cooling device.

17. The connector assembly according to claim 16, wherein the pigtail comprises a squeezable connection line.

18. The connector assembly according to claim 17, wherein the squeezable connection line of the pigtail merges into a hollow needle.

19-36. (canceled)

37. An ablation device comprising:
a cable assembly having
a. a coaxial cable comprising an inner conductor, encompassed by a dielectric layer, encompassed by an outer conductor;
b. a first cooling line;
c. a second cooling line;
d. a thermal conductive layer in a cross-sectional view encompassing the coaxial cable and the first cooling line in a circumferential manner, configured to reduce a thermal peak caused by the coaxial cable when transferring energy, by distributing thermal energy around the coaxial cable and the first cooling line in a circumferential manner.
a handle arranged at a distal end of the cable assembly and configured to interconnect to a catheter; and
a connector assembly arranged at a proximal end of the cable assembly, the connector assembly having
a housing with a therein arranged electrical connector for connecting the coaxial cable to the generator and
a breakout section with a pigtail for connecting at least the first cooling line to the cooling device.
